(19) United States
(12) Patent Application Publication
(10) Pub. No.: US 2014/0243598 A1
(43) Pub. Date: Aug. 28, 2014

(54) OPTICAL PROBE DELIVERY AND RETRIEVAL SYSTEMS AND METHODS

(71) Applicant: CORNING INCORPORATED, CORNING, NY (US)

(72) Inventors: Michael Lucien Genier, Horseheads, NY (US); Paul Michael Then, Victor, NY (US); Peter Gerard Wigley, Corning, NY (US)

(73) Assignee: CORNING INCORPORATED, CORNING, NY (US)

(21) Appl. No.: 14/180,470

(22) Filed: Feb. 14, 2014

Related U.S. Application Data
(60) Provisional application No. 61/768,855, filed on Feb. 25, 2013.

(51) Int. Cl. A61B 1/273; A61B 1/00

(52) U.S. Cl. CPC A61B 1/2736; A61B 1/00154
USPC 600/114; 600/178

(57) ABSTRACT

Optical probe delivery and retrieval systems and methods are disclosed. The optical probe includes a dissolvable capsule that contains micro-components that are substantially smaller than the size of the capsule. The capsule is allowed to dissolve in the stomach after imaging data is collected in the esophagus. Thus, only the optical fiber tether and the relatively small micro-components attached thereto need be retrieved. Delivery devices that facilitate comfortable delivery and retrieval of the optical probe are also disclosed.

### CROSS-REFERENCE TO RELATED APPLICATIONS

This application claims the benefit of priority under 35 U.S.C. §119 of U.S. Provisional Application Ser. No. 61/768,855 filed on Feb. 25, 2013 the contents of which are relied upon and incorporated herein by reference in their entirety.

### FIELD

The present disclosure relates generally to the use of optical probes, and in particular to systems and methods for gastro intestinal (e.g., esophageal) delivery and retrieval of optical probes.

### BACKGROUND

Optical probes are used in the medical field to capture in vivo high-resolution images. One form of in vivo imaging is based on optical coherence tomography or OCT, which is described in U.S. Pat. No. 5,321,501 and U.S. Patent Application Publication No. 2009/0198125, which are incorporated by reference herein. OCT systems can work in the time domain or the frequency domain. Frequency domain OCT is also called optical frequency domain imaging or OFDI.

FIG. 1A is a schematic diagram of a basic in vivo imaging system 10, which includes an optical probe 20, an optical fiber tether 40 operably attached to the optical probe, and an image processing system 46. FIG. 1B is a close-up elevated view of an example optical probe 20. The optical probe 20 contains a number of micro-components, including an optical transceiver 24 and a scanning support member 25, which allows imaging radially around a central axis. The optical probe 20 also includes an elongate capsule 22 that is transparent to the operating wavelength of light and that is about 1 inch long and up to 0.75 inch wide.

The optical transceiver 24 is configured to emit and receive light. In an example, optical transceiver 24 emits light 26 (“emitted light”) and receives sample light 26S from the sample, namely, the esophagus.

In one type of probe 20, the support member 25 rapidly rotates about the axis of elongate capsule 22 so that emitted light 26 from optical transceiver 24 is transmitted through the capsule over 360° and into or onto a sample. The optical transceiver 24 also collects the sample light 26S from the sample in response to being irradiated by emitted light 26. The collected sample light 26S represents image data. This image data is transmitted by optical fiber tether 40 to image processing system 46 for image processing, analysis and storage.

The current state of the art probe 20 is relatively large compared to a conventionally sized pill. Furthermore, capsule 22 is made of glass, plastic or other substance designed to withstand exposure to esophageal and digestive fluids. For esophageal imaging, optical probe 20 must be swallowed and then later mechanically pulled back up the throat and out of the mouth. This procedure can be quite uncomfortable. There is therefore a need for improved systems and methods for delivering and retrieving the optical probe as part of an in vivo esophageal imaging procedure that can make a patient more comfortable during the procedure.

### SUMMARY

An aspect of the disclosure is an optical probe for collecting imaging data from a gastro-intestinal organ (e.g., an esophagus) of a person. The optical probe includes micro-components configured to emit light and receive sample light, wherein the sample light is representative of imaging data. The optical probe also includes a capsule having a wall that is substantially transparent to the emitted light and the sample light and that is configured to not substantially distort the imaging data, wherein the wall defines a capsule interior that contains the micro-components. The capsule is dissolvable when subjected to at least one dissolving fluid fluid.

Another aspect of the disclosure is a delivery device for delivering to a gastro-intestinal organ (e.g., an esophagus) of a person an optical probe having a back end portion and an optical fiber tether attached to the back end. The delivery device includes a delivery tube section sized to pass the optical fiber tether, and a holding feature at one end of the delivery tube section that is configured to accommodate at least a back portion of the optical probe.

Another aspect of the disclosure is a delivery device for delivering to a gastro-intestinal organ (e.g., an esophagus) of a person an optical probe having a back end portion and an optical fiber tether attached to the back end. The device includes a delivery tube section sized to pass the optical fiber tether, and a bulbous member through which the delivery tube passes having front and back ends. The delivery tube extends from both the front and back ends of the bulbous member.

Another aspect of the disclosure is a method of deploying to a gastro-intestinal organ (e.g., an esophagus) of a person through their mouth and throat an optical fiber tether and an optical probe that contains micro-components. The method includes introducing the optical probe and optical fiber tether into the mouth. The method also includes leading the optical probe and optical fiber tether through the throat and down the esophagus with the assistance of peristalsis until the optical probe reaches the stomach. The method also includes allowing the capsule to dissolve in the stomach, and then pulling the optical fiber tether and the micro-components back up through the esophagus, through the throat and out of the mouth.

Additional features and advantages will be set forth in the Detailed Description that follows and in part will be readily apparent to those skilled in the art from the description or recognized by practicing the embodiments as described in the written description and claims thereof, as well as the appended drawings. It is to be understood that both the foregoing general description and the following Detailed Description are merely exemplary and are intended to provide an overview or framework for understanding the nature and character of the claims.

### BRIEF DESCRIPTION OF THE DRAWINGS

The accompanying drawings are included to provide a further understanding and are incorporated in and constitute a part of this specification. The drawings illustrate one or more embodiment(s) and together with the Detailed Description serve to explain principles and operation of the various embodiments. As such, the disclosure will become more fully understood from the following Detailed Description, taken in conjunction with the accompanying Figures, in which:

FIG. 1A is a schematic diagram of an example prior art in vivo imaging system;

FIG. 1B is a close-up elevated view of an example prior art optical probe used in the in vivo imaging system of FIG. 1A;

FIG. 2A is a schematic diagram of an example in vivo imaging system according to the disclosure that utilizes the optical probe as disclosed herein, wherein the optical probe has a dissolvable capsule;

FIG. 2B is a side view of an example optical probe according to the disclosure and that is suitable for use in the in vivo imaging system of FIG. 2A;

FIG. 3 is a schematic diagram of the upper portion of a person's digestive system, showing the mouth, the throat, the esophagus and a portion of the stomach and illustrating an example of how the optical probe and tether are deployed to travel down the esophagus to collect in vivo imaging data;

FIG. 4 is similar to FIG. 2B and shows the optical probe residing in the esophagus of the person undergoing an esophageal imaging procedure;

FIG. 5A is similar to FIG. 3 and shows the optical probe in the stomach, where the dissolvable capsule is subjected to stomach fluid;

FIG. 5B is similar to FIG. 5A, except that the stomach fluid has dissolved the dissolvable capsule, leaving only the micro-components and the optical fiber tether;

FIG. 6 is similar to FIG. 4, except that it shows the dissolvable capsule having been dissolved, leaving the optical fiber tether and the micro-components to be retrieved by pulling on the optical fiber tether;

FIG. 7A is an elevated view of an example embodiment of an optical probe delivery device that has the shape of a lollypop;

FIG. 7B is a longitudinal cross-sectional view of the delivery device of FIG. 7A, showing the optional ferrule that slidingly fits within the delivery tube section and that can be used as a plunger to push the optical probe;

FIG. 7C is a side view of an example delivery device similar to that shown in FIGS. 7A and 7B, illustrating how the plunger can extend from the front end of the bulbous member;

FIG. 7D is a close-up cross-sectional view of a person, showing how the lollypop delivery device is used to facilitate delivery of the optical probe into the esophagus;

FIG. 8 is a cross-sectional view of an example embodiment of a delivery device wherein the delivery-tube-section front end has a holding feature configured to hold the optical probe;

FIG. 9 is a cross-sectional view of an example embodiment of a delivery device that consists of just the delivery tube section;

FIG. 10 is an elevated view of an example embodiment of a delivery device that includes a delivery tube section and a bulbous member through which the delivery tube section passes; and

FIG. 11 is similar to FIG. 7D and shows how the delivery device of FIG. 10 can be used to facilitate delivery of the optical probe into the esophagus.

### DETAILED DESCRIPTION

Reference is now made in detail to various embodiments of the disclosure, examples of which are illustrated in the accompanying drawings. Whenever possible, the same or like reference numbers and symbols are used throughout the drawings to refer to the same or like parts. The drawings are not necessarily to scale, and one skilled in the art will recognize where the drawings have been simplified to illustrate the key aspects of the disclosure.

The claims as set forth below are incorporated into and constitute part of this Detailed Description.

The entire disclosure of any publication or patent document mentioned herein is incorporated by reference.

Cartesian coordinates are shown in some of the Figures for the sake of reference and are not intended to be limiting as to direction or orientation.

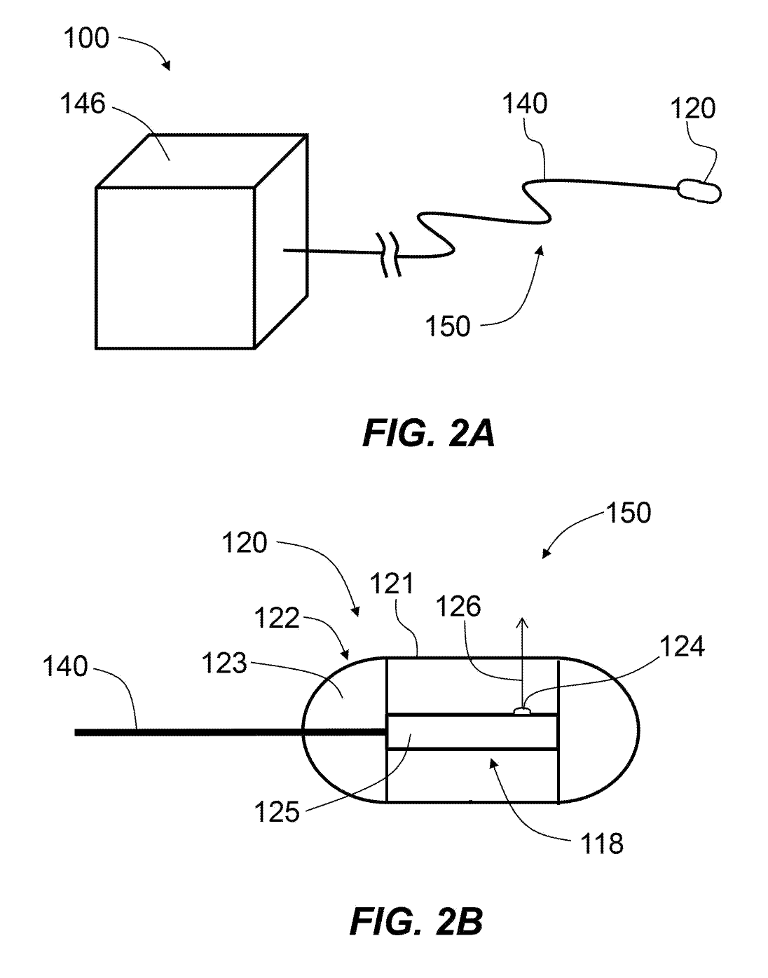

FIG. 2A is a schematic diagram of an example in vivo imaging system 100 that utilizes an optical probe 120 as disclosed herein. FIG. 2B is a schematic cross-sectional view of an example embodiment of optical probe 120. The in vivo imaging system 100 includes optical probe 120 and an optical fiber tether 140, which together form an optical probe assembly 150. The optical fiber tether 140 is optically connected to an image processing system 146 that is configured to receive and process image data from optical probe 120.

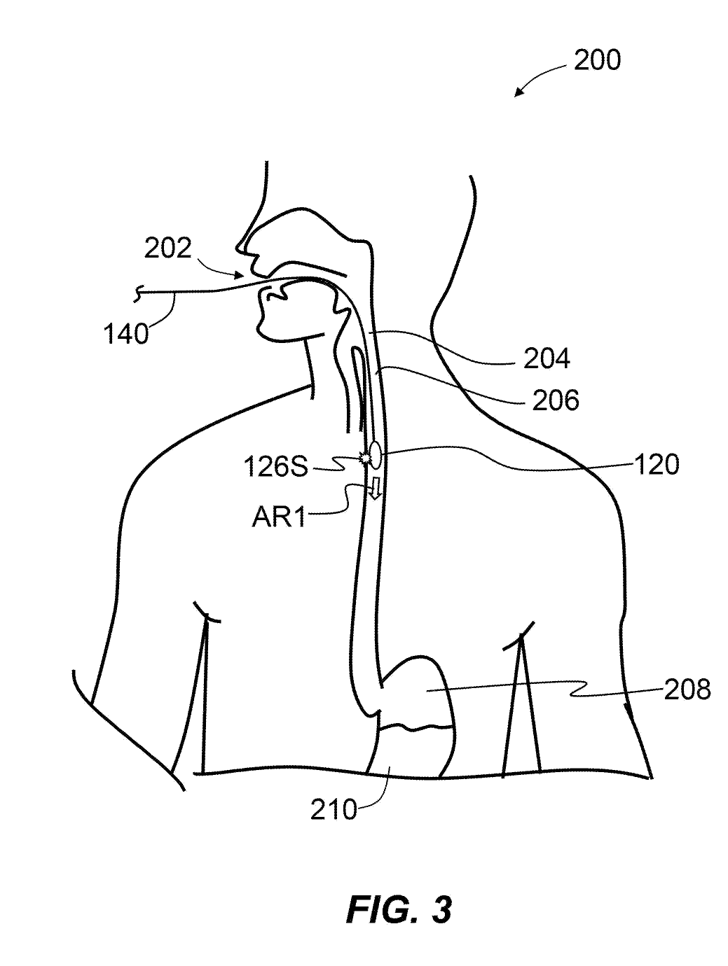

FIG. 3 is a schematic diagram of the upper portion of the digestive system of a person 200. The person's mouth is indicated by 202, the throat by 204, the esophagus by 206 and the stomach by 208. FIG. 3 illustrates an example of how optical probe 120 and tether 140 are deployed to travel down esophagus 206 in the process of collecting imaging data.

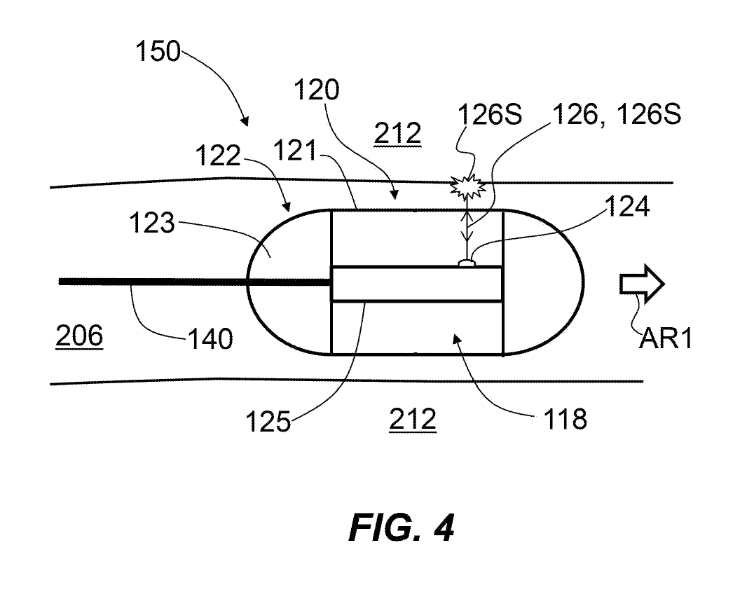

FIG. 4 is similar to FIG. 2B and shows optical probe 120 residing in esophagus 206 of person 200 undergoing an imaging procedure. Esophagus 206 is defined by an esophageal wall 212. The optical probe 120 is optically connected to optical fiber tether 140. The optical probe 120 is introduced into esophagus 206 by virtue of person 200 placing the optical probe in their mouth 202 and swallowing it like a pill. Peristalsis carries optical probe 120 down toward stomach 208, as indicated by arrow AR1. The optical probe 120 captures imaging data as it travels down esophagus 206 by illuminating the esophagus with emitted light 126 and capturing sample light 1265. Imaging data may also be captured as optical probe 120 is made to travel back up esophagus 206, e.g., by not letting the probe reach stomach 208 on its downward travel for reasons explained below.

The sample light 126S may be scattered light, fluorescent light, reflected light, etc. The emitted light 126 has an operating wavelength λ₀, which in an example is an infrared wavelength. The sample light 126S has a sample wavelength λ_{S}, which may be the same as or different from the operating wavelength λ₀ of emitted light 126.

The optical probe 120 includes a dissolvable capsule 122 having a wall 121 that defines an interior 123 that includes one or more micro-components (e.g., optical, mechanical and electrical micro-components) generally denoted as 118. In an example, capsule 122 is elongate and includes a cylindrical center section wherein wall 121 is cylindrical and is thus curved. In an example embodiment, micro-components 118 include a support member 125 and an optical transceiver 124 operably supported by the support member. In an example, support member 125 is rotatable. The optical transceiver 124 emits light 126 and receives sample light 126S. The sample light 126S is representative of imaging data, i.e., the sample light is sent to image processing system 146 to be processed to form images of the esophagus.

In an example, optical fiber tether 140 includes a central optical fiber surrounded by a drive mechanism (not shown) configured to rapidly rotate support member 125 and thus the micro-optical elements 118 supported thereby. In an example, the drive mechanism includes a fine wire torque coil encased in a polymer sheath. Optical fiber tether 140 can have other configurations to facilitate the image data collection process, and the systems and methods disclosed herein are not dependent upon the specific type of optical fiber tether 140 employed.

The optical transceiver 124 and support member 125 typically each comprise a number of additional micro-components that are known in the art. Thus, only an end portion of optical transceiver 124 and a modular support member 125 are shown by way of example and for ease of illustration.

The one or more micro-components 118 are substantially narrower than dissolvable capsule 122. For example, dissolvable capsule 122 may be 1 inch long and up to 0.75 inch wide, while micro-components 118 may be only about 3 mm or 4 mm wide. The dissolvable capsule 122 serves the purpose of providing a liquid-free environment for micro-components 118 with a controlled working distance and otherwise controlled optical environment between the capsule wall and the micro-components. For example, dissolvable capsule 122 allows for the rotation of support member 125 and also preserves the imaging characteristics of optical probe 120. For example, optical transceiver 124 is configured to image through curved wall 121 of dissolvable capsule 122.

Once the imaging data is collected, there is no longer any need for capsule 122. Yet, in the prior art, this capsule is designed to withstand exposure to in vivo fluids and is retrieved intact, often to the substantial discomfort of the person on whom the procedure is being carried out.

The dissolvable capsule 122 is substantially transparent to the operating wavelength λ₀ and the sample wavelength λ_{S} so that the in vivo imaging can be performed through wall 121, as shown in FIG. 4. Unlike the prior art, dissolvable capsule 122 dissolves when exposed to one or more in vivo fluids, such as digestive fluids, e.g., saliva (which has a pH of about 6) and stomach fluid (which has a pH of about 3.5). In an example, dissolvable capsule 122 dissolves more slowly when exposed to esophageal fluids (e.g., saliva) than when exposed to stomach fluid.

In an example embodiment, dissolvable capsule 122 comprises at least one of sugar, salt, cellulose, sucrose, starch and other known digestible and in vivo dissolvable substances. In an example, the dissolvable substance is flavored. In another example, the dissolvable substance includes a topical anesthetic that serves to numb the throat as optical probe 120 and optical fiber tether 140 travel down the throat and into esophagus 206.

In an example, dissolvable capsule 122 is operably attached to optical fiber tether 140. In an example embodiment, dissolvable capsule 122 is designed so that it separates from optical fiber tether 140 before it completely dissolves, and then completely dissolves after the separation. For example, optical probe 120 may be deployed as described above so that it travels down esophagus 206 as it collects imaging data. As optical probe 120 travels down esophagus 206, dissolvable capsule 122 dissolves at a slow rate so that the capsule generally maintains its structural integrity during the imaging data collection process.

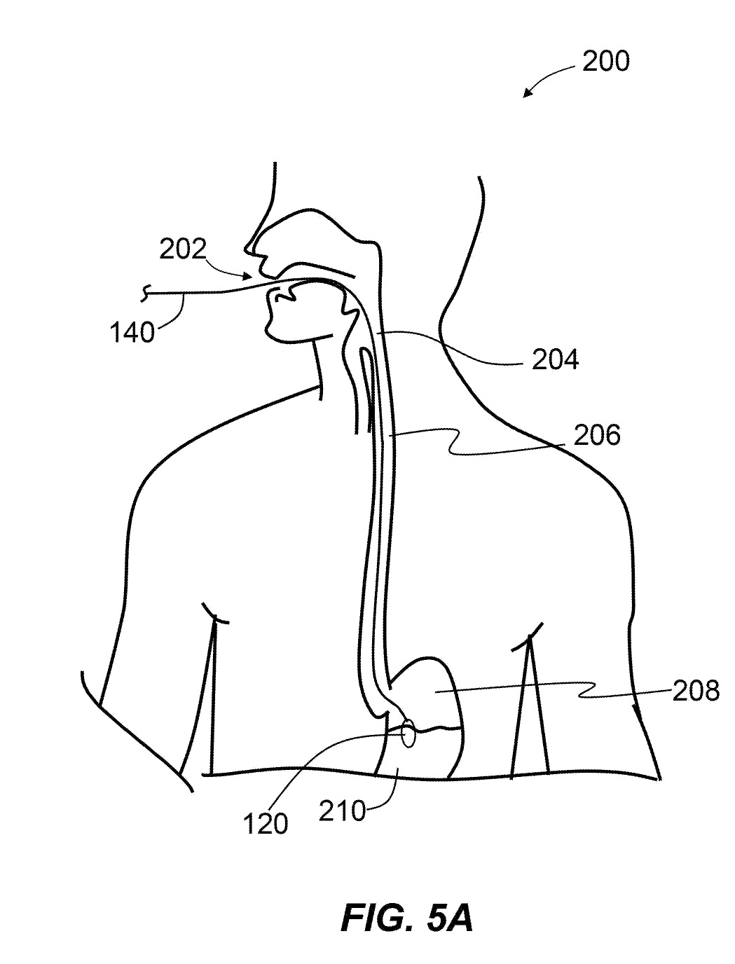

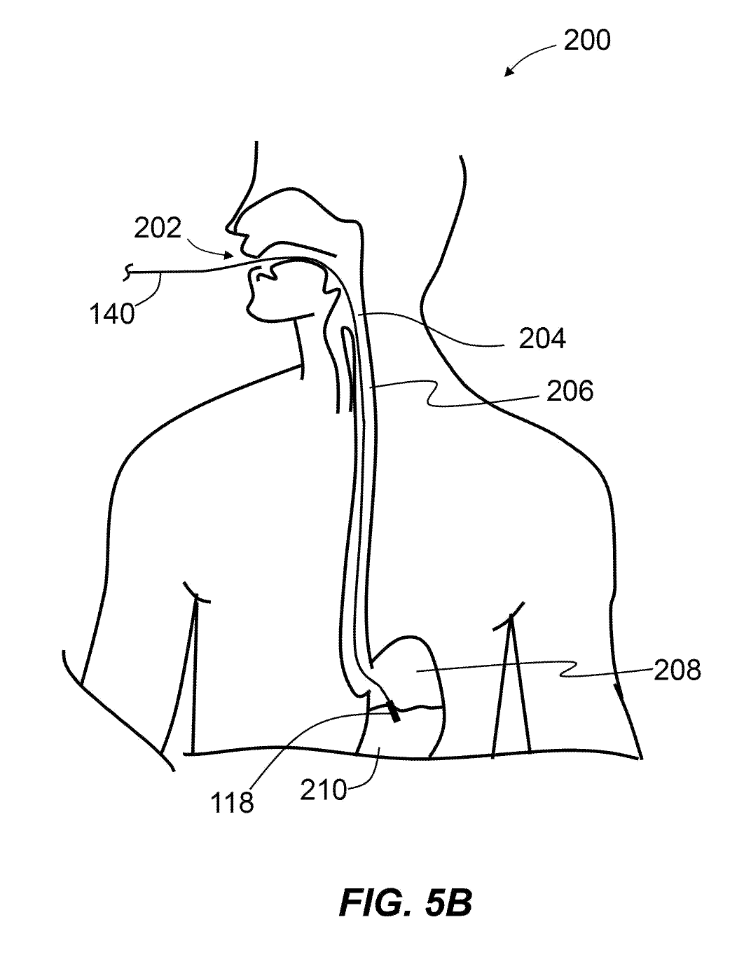

With reference to FIG. 5A, in an example optical probe 120 is then introduced into stomach 208, which includes stomach fluid 210. The stronger stomach fluid 210 more quickly dissolves dissolvable capsule 122, leaving just micro-components 118, as shown in FIG. 5B and FIG. 6. In an example embodiment, person 200 can consume a dissolving substance prior to carrying out the esophageal imaging procedure, wherein the consumed dissolving substance then resides in the stomach and adds to (i.e., mixes with) the existing stomach fluid 210. The consumed dissolving substance can have one or more select properties, such as acidity, that make stomach fluid 210 stronger so that capsule 122 dissolves faster (i.e., increases the stomach fluid's dissolving ability). The dissolving substance can be delivered as a fluid via drinking, in capsule or pill form via swallowing, etc.

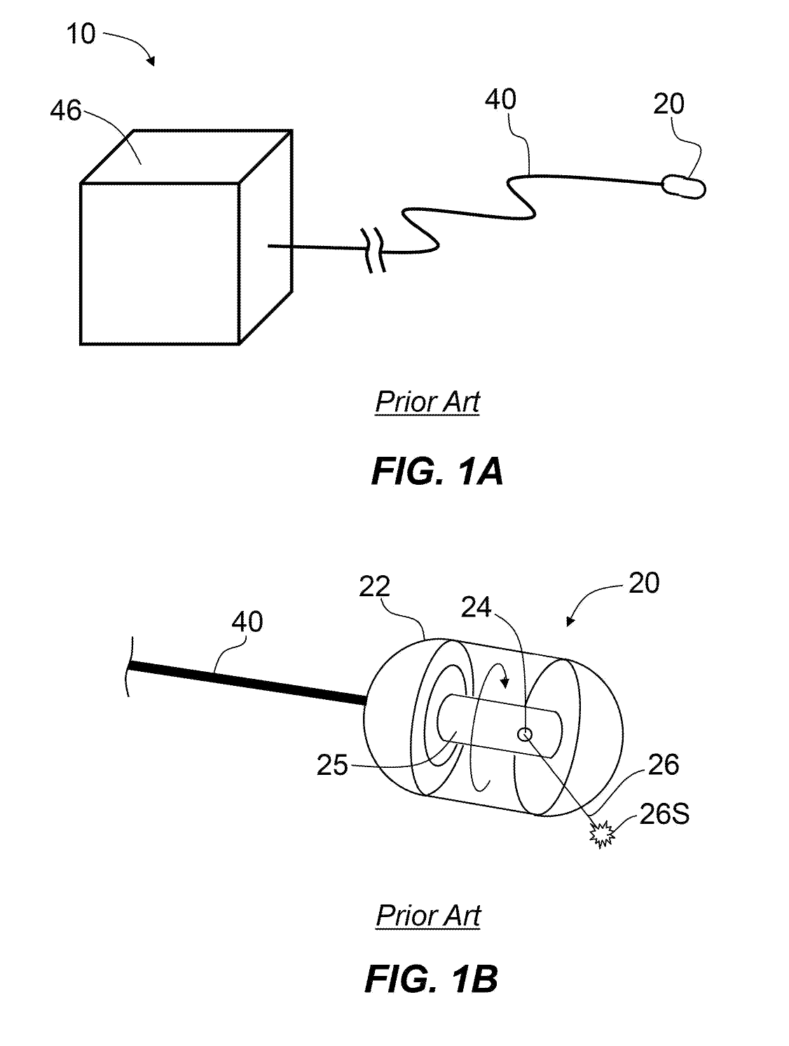

Because micro-components 118 are substantially narrower than dissolvable capsule 122, retrieval of optical fiber tether 140 and the micro-components is more comfortable for person 200 than retrieval of a non-dissolvable capsule such as capsule 22 of the prior art (see FIGS. 1A and 1B). FIG. 6 shows optical fiber tether 140 and micro-components 118 being drawn back out of esophagus 206 in the direction indicated by arrow AR2.

In an example, dissolvable capsule 122 is formed via a molding process whereby the dissolvable substance is heated to its melting point and then poured into a mold with adequate thermal controls to cool the capsule. In another example, the dissolvable substance is machined and polished from a block of dissolvable substance to form dissolvable capsule 122. In another example, the dissolvable substance is extruded in the form of a tube and subsequently shaped into a capsule using a mechanical or heating process to form dissolvable capsule 122.

In an example, dissolvable capsule 122 is made of two or more pieces configured to fit together to encapsulate micro-components 118. In an example, the dissolvable substance can be used to bond dissolvable capsule 122 to optical fiber tether 140 in a manner that will allow the dissolvable capsule to separate from the optical fiber tether when the dissolvable substance is exposed to stomach fluid 210. In an example, the dissolvable substance can be configured so that the dissolvable capsule 122 is time-released from optical fiber tether 140.

Regardless of the method used to form dissolvable capsule 122, it needs to be performed in a manner that avoids substantial optical distortion in the portion of capsule wall 121 through which emitted light 26 and sample light 26S pass.

### Delivery Device

An aspect of the disclosure is directed toward a device for facilitating the delivery (insertion) of optical probe 120 into esophagus 206. The use of a delivery device has a number of advantages, including limiting the interaction of the sensitive optical probe 120 with mouth 202 to avoid person 200 accidently damaging the optical probe. Another advantage is making the procedure more comfortable for person 200 through the more accurate placement and alignment of optical probe 120.

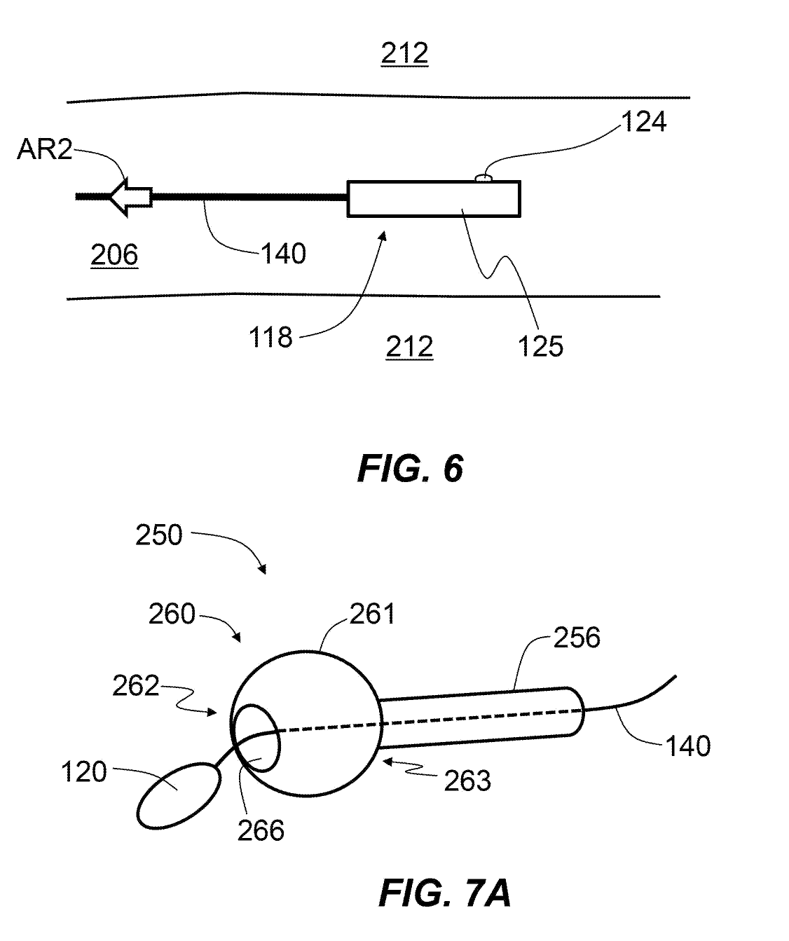

FIG. 7A is an elevated view of an example delivery device 250, while FIG. 7B is a longitudinal cross-sectional view of the delivery device. The delivery device 250 includes a delivery tube section 256 and a holding feature 260 at one end of the delivery tube section. The holding feature 260 is generally configured to hold or otherwise support at least a portion of optical probe 120. The delivery device 250 is configured to be placed into mouth 202 of person 200 and then held therein by the person.

In the example delivery device 250 of FIGS. 7A and 7B, holding feature 260 is in the form of a bulbous member 261 so that the delivery device has the form of a lollypop. The bulbous member 261 has a front end 262 and a back end 263, with delivery tube section 256 interfacing the bulbous member at the back end. The bulbous member 261 includes a cavity 266 that is open at front end 262. The bulbous member 261 can be any bulbous shape, including spherical, ellipsoidal, oblate and the like.

The cavity 266 of bulbous member 261 is sized to accommodate at least a back end portion 127 of probe 120. The optical fiber tether 140 attached to capsule 122 passes through delivery tube section 256. In an example embodiment, delivery device 250 optionally includes a plunger 270 that slidingly fits within delivery tube section 256 and that includes an end 272 that can contact back end portion 127 of optical probe 120. In an example, end 272 of plunger 270 is configured to engage or capture back end portion 127 of optical probe 120. Because plunger 270 can slide within delivery tube section 256, it can be used to push optical probe 120 out of holding feature 260 and into mouth 202 and then into throat 204.

In the example of delivery device 250 illustrated in FIG. 7B, a dissolvable substance 274 may be employed to retain optical probe 120 in holding feature 260. In an example, dissolvable substance 274 may be any edible substance that readily dissolves in a dissolving fluid. Example dissolvable substances include starch, cellulose, sucrose, salt, sugar, etc. An example in vivo dissolving fluids is saliva. An example non-in-vivo dissolving fluid is an acidic fluid that is introduced into the mouth, e.g., as an acidic juice. The dissolvable substance 274 then dissolves when placed in mouth 202 and subjected to the dissolving fluid therein, thereby allowing optical probe 120 to be released into the mouth and down throat 204.

FIG. 7C is a side view of an example delivery device 250 similar to that shown in FIGS. 7A and 7B. In the example of FIG. 7C, plunger 270 is arranged so that end 272 of the plunger can protrude beyond front end 262 of bulbous member 261 and can be extended or retracted relative to the front end by sliding the plunger back and forth within delivery tube section 256. This configuration allows for delivery tube section 256 to extend into throat 204, thereby allowing optical fiber tether 140 to pass through the throat without touching the throat wall. Person 200 will thus have a reduced sensation of the moving optical fiber tether during the procedure.

FIG. 7D is a close-up cross-sectional view of person 200 showing how an example delivery device 250 such as that shown in FIGS. 7A through 7C can be used to deploy optical probe 120 into esophagus 206. The person 200 can bite down on the portion of delivery tube section 256 that extends from back end 263 of bulbous member 261 to secure delivery device 250 within mouth 202. The lollypop configuration of delivery device 250 allows person 200 to insert bulbous member 261 into their mouth 202 and hold it fixed therein, e.g., by clamping down on the bulbous member or delivery tube section 256 with their teeth, by sucking on the bulbous member, or by pressing the bulbous member to the roof of their mouth with their tongue. The person 200 can also facilitate the delivery of optical probe 120 by sucking on bulbous member 261 so that the optical probe travels down throat 204 and then esophagus 206 via peristalsis.

In an example, bulbous member 261 is flavored. In an example, bulbous member 261 is at least partially made of sugar or conventional lollypop constituents to make the insertion process of optical probe 120 more comfortable. For example, bulbous member 261 can be made of a solid inedible substance (e.g., plastic) and include an edible coating 276, as shown in FIG. 7B.

A flavored bulbous member 261 will also tend to cause person 200 to generate saliva, which serves as lubrication for facilitating the passage of optical probe 120 down throat 204 and esophagus 206. In an example, bulbous member 261 is provided with a mild oral anesthetic to reduce any uncomfortable sensations during the insertion process. The bulbous member 261 can also be made aromatic.

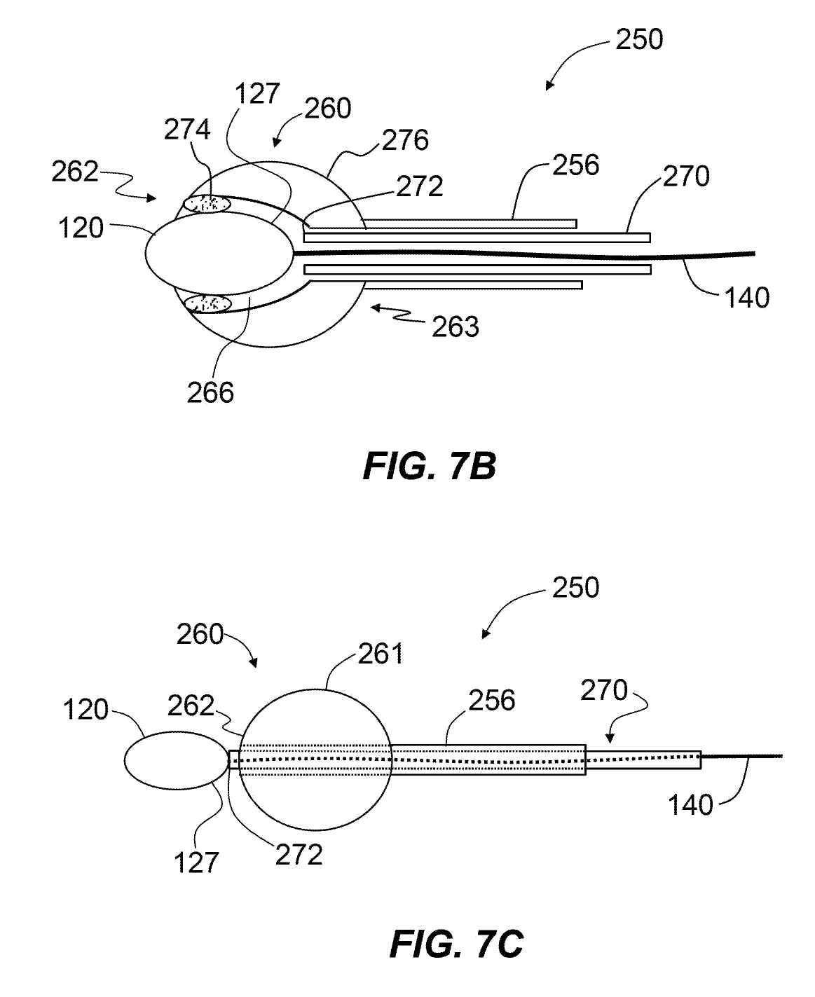

FIG. 8 shows a cross-sectional view of another example of delivery device 250, wherein holding feature 260 comprises a cup 280 that is sized to accommodate the end portion 127 of optical probe 120 to which optical fiber tether 140 is attached. In this example, holding feature 260 may be formed by expanding the end of delivery tube section 256. In an example, cup 280 has a shape that is complimentary to back end portion 127 of optical probe 120.

In an example, cup 280 receives back end portion 127 of optical probe 120, which can be held therein by providing tension on optical fiber tether 140. In an example, cup 280 can be provided as an attachment to delivery tube section 256. In FIG. 8, optional plunger 270 is not shown for ease of illustration but can be included and employed in the same manner as described above.

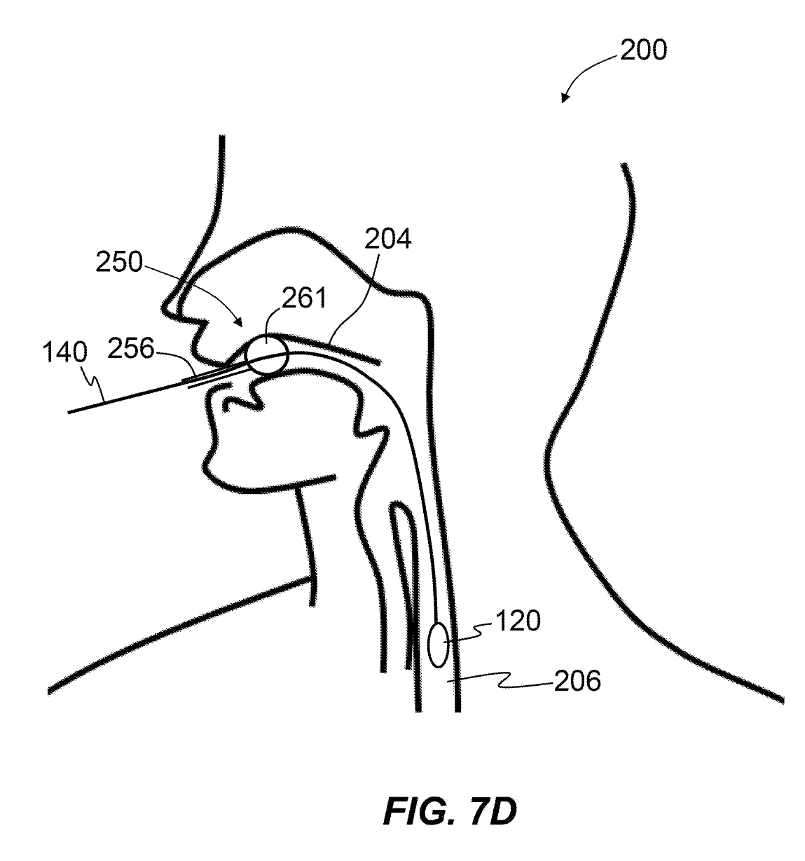

FIG. 9 is similar to FIG. 8 and shows an even simpler example of delivery device 250 that consists of just delivery tube section 256. In this embodiment, optical probe 120 is held at one end of delivery tube section 256 by providing tension on optical fiber tether 140 until the optical probe is ready to be deployed. In FIG. 9, optional plunger 270 is not shown for ease of illustration but can be included and employed in the same manner as described above.

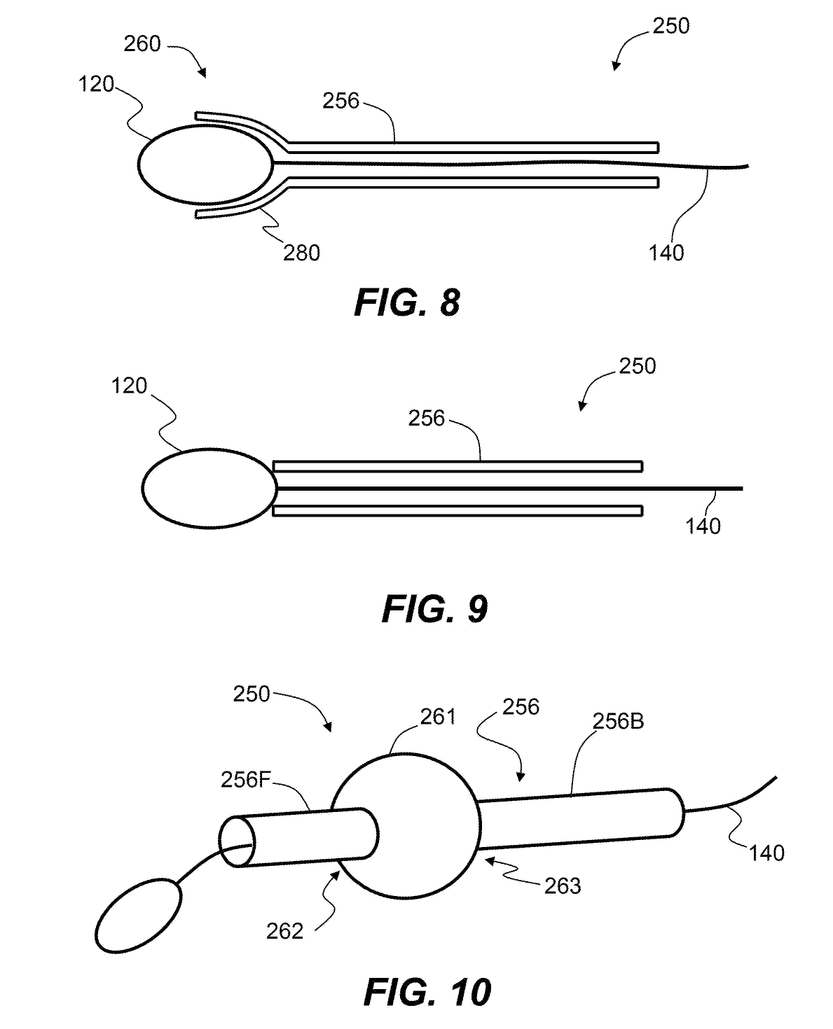

FIG. 10 shows a modified version of delivery device 250 of FIG. 9 wherein delivery tube section 256 extends through front end 262 of bulbous member 261, which in an example is otherwise solid. Alternatively, plunger 270 extends from front end 262 of bulbous member 261, as described above and as illustrated in FIG. 7C. The bulbous member 261 can have one or more of the above-described attributes of having a flavor, an aroma, an anesthetic, an edible coating 276, etc. The portion of delivery tube section 256 that extends from front end 262 of bulbous member 261 is referred to hereinafter as front delivery tube section 256F and the portion that extends from back end 263 of the bulbous member is referred to hereinafter as back delivery tube section 256B. As noted above, in an example, front delivery tube section 256F is defined by plunger 270, which can be extended or retracted relative to front end 262 of bulbous member 261.

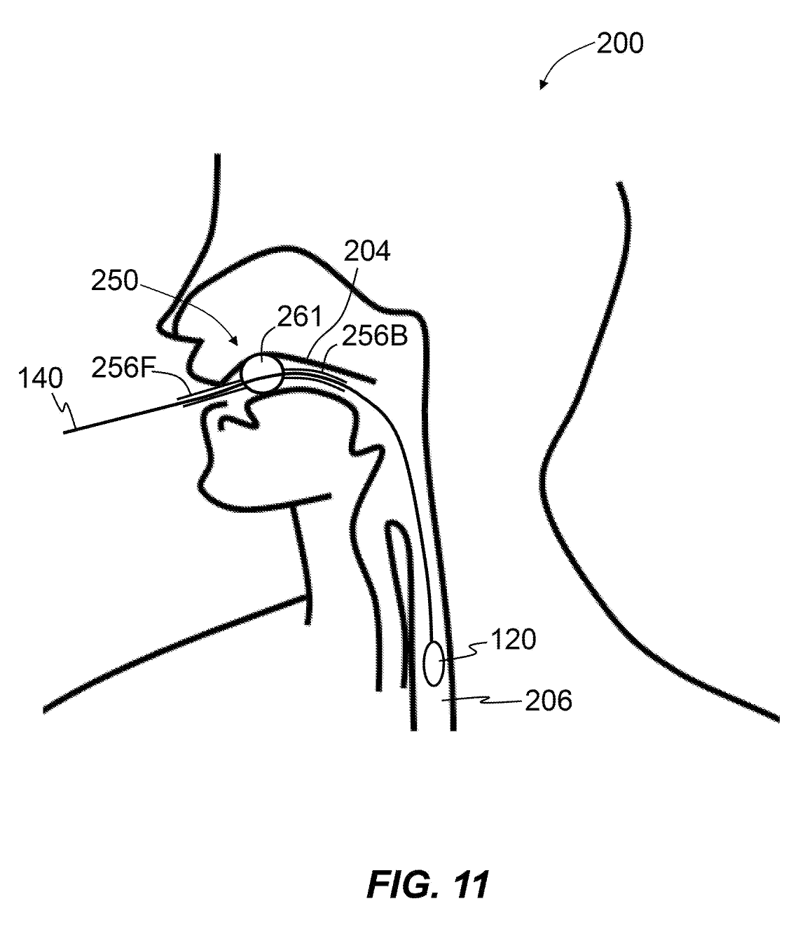

FIG. 11 is similar to FIG. 7D and illustrates how the example delivery device 250 of FIG. 10 is used to deploy optical probe 120 into esophagus 206. The front delivery tube section 256F serves to guide optical probe 120 down throat 204 of person 200 during the insertion procedure while both the front delivery tube section and back delivery tube section 256B serve to guide optical fiber tether 140. In an example, front delivery tube 256F is flexible so that it can conform to the curvature of throat 204. The throat 204 can close down on front delivery tube section 256F, which secures the delivery tube section in place while also allowing optical fiber tether 140 to be inserted down esophagus 206. The person 200 can bite down on front delivery tube section 256F to secure delivery device 250 in place in mouth 202.

An aspect of the disclosure is a method of deploying optical probe 120, which contains micro-components 118, and optical fiber tether 140 into esophagus 206 of person 200 through their mouth 202 and throat 204. The method includes introducing optical probe 120 and optical fiber tether 140 into mouth 202 of person 200. The method also includes leading optical probe 120 and optical fiber tether 140 through throat 204 and down esophagus 206 with the assistance of peristalsis until the optical probe reaches stomach 208. The method also includes allowing capsule 122 to dissolve in stomach 208, and then pulling optical fiber tether 140 and micro-components 118 back up through esophagus 206, through throat 204 and out of mouth 202.

Thus, according to some embodiments

It will be apparent to those skilled in the art that various modifications to the preferred embodiments of the disclosure as described herein can be made without departing from the spirit or scope of the disclosure as defined in the appended claims. Thus, the disclosure covers the modifications and variations provided they come within the scope of the appended claims and the equivalents thereto.

What is claimed is:

1. An optical probe for collecting imaging data from an internal gastro-intestinal organ of a person, comprising:
micro-components configured to emit light and receive sample light, wherein the sample light is representative of imaging data;
a capsule having a wall that is substantially transparent to the emitted light and the sample light and that is configured to not substantially distort the imaging data, wherein the wall defines a capsule interior that contains the micro-components; and
wherein the capsule is dissolvable when subjected to at least one dissolving fluid.

2. The optical probe according to claim 1, wherein the at least one dissolving fluid includes saliva.

3. The optical probe according to claim 1, wherein the capsule comprises at least one of: starch, cellulose, sucrose, salt, sugar, a flavored substance, at least a layer of an edible substance, an aromatic substance, and a local anesthetic.

4. The optical probe according to claim 1, further comprising an optical fiber tether optically connected to the optical transceiver through the support member.

5. The optical probe according to claim 4, wherein the capsule is configured to separate from either the optical fiber tether or the support member when subjected to the dissolving fluid.

6. The optical probe according to claim 5, wherein the capsule is attached to the optical fiber tether via one or more substances that are dissolvable by the dissolving fluid.

7. A delivery device for delivering in an gastro-intestinal organ of a person an optical probe having a back end portion and an optical fiber tether attached to the back end portion, comprising:
a delivery tube section sized to pass the optical fiber tether; and at least one of the following:
(a) a holding feature at one end of the delivery tube section and configured to accommodate at least the back end portion of the optical probe;
(b) bulbous member having front and back ends and through which the delivery tube section passes, and wherein the delivery tube section extends from both the front and back ends of the bulbous member.

8. The delivery device according to claim 7, wherein the holding feature comprises:
(i) the bulbous member sized to fit into a mouth of the person; or
(ii) a bulbous member sized to fit into the mouth of the person, the bulbous member having a front end, and wherein the plunger end extends through the front end of the bulbous member/

9. The delivery device according to claim 8, wherein the bulbous member at least partially comprises at least one of: a flavored substance, a layer of an edible substance, an aromatic substance and a local anesthetic.

10. The delivery device according to claim 7, further comprising a plunger that slidingly fits within the delivery tube section and that has an end that contacts the optical probe.

11. The delivery device according to claim 10, wherein the holding feature comprises a bulbous member sized to fit into the mouth of the person, the bulbous member having a front end, and wherein the plunger end extends through the front end of the bulbous member.

12. The delivery device according to claim 8, wherein the delivery tube extends through the bulbous member.

13. A delivery device for delivering to an internal organ of a person an optical probe having a back end portion and an optical fiber tether attached to the back end portion, comprising:
a delivery tube section sized to pass the optical fiber tether; and
a bulbous member having front and back ends and through which the delivery tube section passes, and wherein the delivery tube section extends from both the front and back ends of the bulbous member.

14. The delivery device according to claim 13, wherein the bulbous member at least partially comprises at least one of: a flavored substance, a layer of an edible substance and a local anesthetic.

15. A method of deploying both an optical probe having a capsule containing micro-components and an optical fiber tether into a person through their mouth and throat, comprising:
introducing the optical probe and optical fiber tether into the mouth;
leading the optical probe and optical fiber tether through the throat and down the esophagus with the assistance of peristalsis until the optical probe reaches the stomach;
allowing the capsule to dissolve in the stomach; and
pulling the optical fiber tether and the micro-components back up through the esophagus, through the throat and out of the mouth.

16. The method according to claim 15, wherein the stomach includes a stomach fluid, and further comprising:
attaching the capsule to the optical fiber tether with a dissolvable substance;
allowing the stomach fluid to dissolve the dissolvable substance, thereby detaching the capsule from the optical fiber tether; and
performing said pulling of the optical fiber tether and the micro-components back up through the esophagus, through the throat and out of the mouth while the capsule dissolves in the stomach fluid.

17. The method according to claim 15, wherein the capsule: (i) comprises at least one of starch, cellulose, sucrose, salt and sugar; and/or (ii) is dissolvable faster in a stomach fluid than in saliva.

18. The method according to claim 15, further comprising using a delivery device to perform the introduction of the optical probe and optical fiber tether into the mouth.

19. The method according to claim 18, wherein the delivery device includes a bulbous member sized to reside in the mouth and a delivery tube, and including introducing the bulbous member into the mouth and feeding the optical fiber tether through the delivery tube and the bulbous member.

20. The method according to claim 19, wherein the bulbous member at least partially comprises at least one of: a flavored substance, a layer of an edible substance and a local anesthetic.
